(19) United States
(12) Patent Application Publication
(10) Pub. No.: US 2003/0211601 A1
(43) Pub. Date: Nov. 13, 2003

(54) Isolated bladder submucosa for tissue reconstruction

(75) Inventor: Anthony Atala, Weston, MA (US)

Correspondence Address: LAHIVE & COCKFIELD, 28 STATE STREET, BOSTON, MA, 02109, US

(73) Assignee: THE CHILDREN'S HOSPITAL, Boston, MA

(21) Appl. No.: 10/458,503

(22) Filed: Jun. 9, 2003

Related U.S. Application Data
(63) Continuation of application No. 08/911,218, filed on Aug. 15, 1997, now abandoned.
(60) Provisional application No. 60/024,029, filed on Aug. 16, 1996.

(51) Int. Cl. A61K 45/00; C12N 5/08

(52) U.S. Cl. 435/325; 424/93.7

(57) ABSTRACT

Methods and materials for tissue reconstruction, repair, and/or augmentation are disclosed. The invention provides isolated bladder submucosa for use in tissue reconstruction. The methods of the invention include the use of isolated bladder submucosa for repair, reconstruction, and/or augmentation of bladder and other organs and tissues.

### CROSS-REFERENCE TO RELATED APPLICATION

This application claims the benefit of priority under 35 U.S.C. 119(e) to co-pending U.S. provisional application Serial No. 60/024,029, entitled “Bladder Augmentation Using Allogenic Bladder Submucosa”, filed Aug. 16, 1996, the entire content of which is hereby incorporated by reference.

### BACKGROUND OF THE INVENTION

Reconstructive surgery has been used for many years for the treatment of congenital tissue defects and for repair of damaged organs and tissues. An ideal material for tissue reconstruction should be biocompatible, able to incorporate into the native tissue without inducing an adverse tissue response, and should have adequate anatomical and functional properties (for example, size, strength, durability, and the like). Although a large number of bio-materials, including synthetic and naturally-derived polymers, have been employed for tissue reconstruction or augmentation (see, e.g., “Textbook of Tissue Engineering” Eds. Lanza, R., Langer, R., and Chick, W., ACM Press, Colorado (1996) and references cited therein), no material has proven satisfactory for use in every application.

For example, in the field of bladder reconstruction, synthetic biomaterials such as polyvinyl and gelatin sponges, polytetrafluoroethylene (Teflon) felt, and silastic patches have been relatively unsuccessful, generally due to foreign body reactions (see, e.g., Kudish, H. G., J. Urol. 78:232 (1957); Ashkar, L. and Heller, E., J. Urol. 98:91 (1967); Kelami, A. et al., J. Urol. 104:693 (1970)). Polymeric materials have been used as “scaffolds” for seeding cells; the seeded scaffolds can be implanted to provide a matrix for the growth of new tissue (see, e.g., Atala, A. et al., J. Urol. 148 (2 Pt 2): 658-62 (1992); Atala, A., et al. J. Urol. 150 (2 Pt 2): 608-12 (1993)). Naturally-derived materials such as lyophilized dura, de-epithelialized bowel segments, and small intestinal submucosa (SIS) have also been proposed for bladder replacement (for a general review, see Mooney, D. et al., “Tissue Engineering: Urogenital System” in “Textbook of Tissue Engineering” Eds. Lanza, R., Langer, R., and Chick, W., ACM Press, Colorado (1996)).

It has been reported that bladders augmented with dura, peritoneum, placenta and fascia contract over time (Kelami, A. et al., J. Urol. 105:518 (1971)). De-epithelized bowel segments demonstrated an adequate urothelial covering for use in bladder reconstruction, but difficulties remain with either mucosal regrowth, segment fibrosis, or both. It has been shown that de-epithelization of the intestinal segments may lead to mucosal regrowth, whereas, removal of the mucosa and submucosa may lead to retraction of the intestinal segment (see, e.g., Atala, A., J. Urol. 156:338 (1996)).

Xenogenous porcine SIS has been used recently with favorable results (e.g., Kropp, B. P. et al, Urology 46:396 (1995)). This biodegradable collagen-rich xenogenic membrane had been previously studied as a potential material for vascular grafts (see, e.g., Hiles et al., J. Biomed. Materials Research 27:139 (1993)). However, SIS may be limited by the maximum size the graft can cover, which may not be sufficient for bladder replacement.

Other problems reported with the use of certain gastrointestinal segments for bladder surgery, including infection, perforation, stone formation, metabolic derangements and instances of tumor development. Formalin-preserved sections of bladder have been used for bladder reconstruction (see, e.g., Tsuji et al., J. Urol. 98:91 (1967)). However, the use of the formalin-preserved material generally did not result in effective long-term treatment.

Polymeric and naturally-derived “scaffolds” have also been used to support the regrowth of bone into bone defects (see, e.g., U.S. Pat. Nos. 5,112,354 and 4,172,128; for a general review, see Yaszemski, M. J.; et al., Biomaterials 17 (2): 175-85 (1996) and references cited therein). Bone-derived collagen implants have been used for bone repair. However, these materials do not always provide the requisite strength, flexibility, or non-immunogenicity needed for long-term repair of bone.

### SUMMARY OF THE INVENTION

The present invention relates to materials and methods for repairing or augmenting tissues. More particularly, the invention relates to methods for tissue reconstruction or repair using bladder submucosa, to methods for preparing bladder submucosa segments suitable for use in tissue reconstruction or repair, and to materials for use in tissue reconstruction or repair.

In one aspect, the invention provides a method for surgically augmenting a tissue of a subject. The method includes the step of augmenting the tissue of the subject with isolated bladder submucosa. The isolated bladder submucosa can be substantially free of cells. The tissue of the subject can be bladder tissue. In a preferred embodiment, the isolated bladder submucosa is allogenic bladder submucosa. In other embodiments, the isolated bladder submucosa is xenogenic bladder submucosa. The subject can be a mammal, including a human. The step of augmenting the tissue of the subject can include surgically grafting the isolated bladder submucosa to the tissue of the subject. In certain embodiments, the isolated bladder submucosa further comprises a growth factor for promoting growth of the tissue.

In another embodiment, the invention provides a method for increasing bladder capacity of a subject having a bladder. The method includes surgically grafting isolated bladder submucosa to the bladder of the subject, such that the bladder capacity is increased.

In another aspect, the invention provides a material for tissue reconstruction or augmentation, the material comprising isolated bladder submucosa. The material can further include an effective amount of a growth factor. In certain embodiments, the material is obtained by microdissection of bladder tissue.

### DETAILED DESCRIPTION OF THE INVENTION

The present invention provides methods and materials for tissue reconstruction and repair. In general, the invention features the use of isolated bladder submucosa for tissue repair and augmentation.

Bladder tissue in vivo contains three principal layers: the submucosal layer, the muscle layer, and the urothelial layer. As used herein, the term “isolated bladder submucosa” refers to bladder submucosa which is substantially free of naturally-occurring urothelial and muscle layers of bladder. In preferred embodiments, isolated bladder submucosa is substantially free of naturally-occurring muscle and urothelial cells. In certain embodiments, isolated bladder submucosa is substantially cell-free. Isolated bladder submucosa is a collagen-rich layer which is substantially non-immunogenic, acellular, and bioresorbable. Commonly-owned copending U.S. patent application entitled “Bladder Submucosa Seeded With Cells For Tissue Reconstruction”, filed on even date herewith, hereby incorporated by reference, describes the use of isolated bladder submucosa, seeded with added cells, for tissue repair and reconstruction.

Isolated bladder submucosa can be obtained, e.g., according to the methods described herein. For example, sections of bladder harvested from a subject can be microdissected to remove the muscle and urothelial layers from the submucosa (e.g., as described in the Example, infra) to produce isolated bladder submucosa, which, in certain embodiments, can be washed, e.g., with phosphate-buffered saline (PBS) to remove extraneous materials, blood, and the like. In certain embodiments, microdissected bladder submucosa can be further treated to ensure that the isolated bladder submucosa preparation is acellular. For example, sections of microdissected bladder submucosa can be placed in distilled water to lyse any remaining cells which adhere to the collagenous submucosal layer. Further treatments, e.g., with deoxyribonuclease (e.g., deoxyribonuclease I) to remove any remaining nucleic acids, can be employed to further ensure that isolated bladder submucosa is cell-free. Such treatments will be routine to one of ordinary skill in the art in light of the teachings herein (see also Sutherland, R. S., et al. J. Urol. 156:571 (1996)).

It will be appreciated that the isolated bladder submucosa can be treated with additives prior to implantation, e.g., to promote the formation of new tissue after implantation. Thus, for example, growth factors, cytokines, extracellular matrix components, and other bioactive materials can be added to the isolated bladder submucosa to promote graft healing and formation of new tissue. Such additives will in general be selected according to the tissue or organ being reconstructed or augmented, to ensure that appropriate new tissue is formed in the engrafted organ or tissue (for examples of such additives for use in promoting bone healing, see, e.g., Kirker-Head, C. A. Vet. Surg. 24 (5): 408-19 (1995) and references cited therein). For example, when isolated bladder submucosa is used to augment vascular tissue, vascular endothelial growth factor (VGEF, see, e.g., U.S. Pat. No. 5,654,273) can be employed to promote the formation of new vascular tissue. Growth factors and other additives can be added in an amount effective to promote the formation of new tissue of a type appropriate to the tissue or organ which is to be repaired or augmented (e.g., by causing or accelerating infiltration of host cells into the graft).

While reference is made herein to augmentation of bladder according to the invention, it will be understood that the methods and materials of the invention are useful for tissue reconstruction or augmentation of a variety of tissues and organs in a subject. Thus, for example, organs or tissues such as bladder, ureter, urethra, renal pelvis, and the like, can be augmented or repaired with isolated bladder submucosa. The materials and methods of the invention further can be applied to the reconstruction or augmentation of vascular tissue (see, e.g., Zdrahala, R. J., J. Biomater. Appl. 10 (4): 309-29 (1996)), intestinal tissues, stomach, cartilage, bone (see, e.g., Laurencin, C. T. et al., J. Biomed. Mater. Res. 30 (2): 133-8 1996), and the like. The term “subject,” as used herein, refers to a mammal, such as a dog, cat, pig, horse, cow, or human, in need of reconstruction, repair, or augmentation of a tissue.

Isolated bladder submucosa can be obtained from whole bladder tissue as described herein. Acellular isolated bladder submucosa is believed to be substantially non-immunogenic. In certain preferred embodiments, isolated bladder submucosa for tissue repair or augmentation is obtained from an animal of the same species as the subject; such tissue is referred to herein as “allogenic” bladder submucosa. However, the nonimmunogenic qualities of isolated bladder submucosa can permit the use of isolated bladder submucosa obtained from a species different from the subject (referred to herein as “xenogenic” isolated bladder submucosa). The use of xenogenic isolated bladder submucosa is especially advantageous when allogenic isolated bladder submucosa is difficult to obtain, e.g., when the subject is a human. Thus, isolated bladder submucosa can be obtained from animals, such as pigs, from which adequate quantities are readily available, for use in repair or augmentation of tissues or organs of a subject of another species. Additionally, xenogenic or allogenic isolated bladder submucosa can be obtained from cadavers.

In a preferred embodiment, the materials and methods of the invention are useful for the reconstruction or augmentation of bladder tissue. Thus, the invention provides treatments for such conditions as bladder exstrophy, bladder volume insufficiency, reconstruction of bladder following partial or total cystectomy, repair of bladder damaged by trauma, and the like.

It has now been found that isolated bladder submucosa can permit the formation of new tissue having a grossly normal cellular organization. For example, as described in the Example, infra, isolated bladder submucosa grafted into bladder served as a “scaffold” for the formation of new bladder tissue within about two months. The new tissue consisted of a urothelial lined lumen surrounded by submucosal tissue and smooth muscle. Moreover, the newly-formed tissue showed evidence of angiogenesis, and nerve growth. Without wishing to be bound by any theory, it is believed that cells from the host animal can infiltrate and grow on the isolated bladder submucosa graft, thereby providing new tissue which is structurally and functionally similar to native tissue, e.g., bladder tissue. It is further believed that the isolated bladder submucosa graft can, in at least some instances, be resorbed into the target organ or tissue; similar results have been observed in the grafting of SIS.

Grafting of isolated bladder submucosa to an organ or tissue to be augmented can be performed according to the methods described herein or according to art-recognized methods. Thus, for example, isolated bladder submucosa can be grafted to an organ or tissue of the subject by suturing the graft material to the target organ, e.g., as described in Example 1, infra. Other methods for attaching a graft to an organ or tissue of the subject (e.g., by use of surgical staples) may also be employed. Such surgical procedures can be performed by one of ordinary skill in the art according to known procedures.

The methods and materials of the invention have been found to be useful in bladder augmentation, as described herein. In a preferred embodiment, isolated bladder submucosa is used for augmentation of bladder, to provide an augmented bladder having a volume (capacity) at least about 20% greater than the pre-augmentation capacity, more preferably at least about 30% greater, 40% greater, 50%, 70% 100% or 200% greater bladder capacity. It has been found that some contraction of the bladder may occur after grafting of isolated bladder submucosa to bladder. Without wishing to be bound by theory, it is believed that such contraction may be due to self-adherence of the grafted material. Accordingly, if desired, the graft site (or the isolated bladder submucosa) can be treated with materials for preventing self-adherence of the graft material, thereby preventing contraction of the grafted bladder and providing increased bladder capacity in the subject. Materials for the prevention of surgical adhesions are commercially available.

### EXAMPLE

Canine bladder tissue was aseptically obtained from sacrificed animals. The bladder tissue was repeatedly rinsed with phosphate buffered saline (PBS). The submucosa was microdissected and isolated from the muscular and serosal layers. The isolated submucosa was thoroughly washed and placed in PBS containing 10% cefazolin. The submucosa was then kept at 4° C. for 6 to 12 months. All segments of allogenic bladder submucosa, measuring 4×5 cm in size, were exposed to UV light for 24 hours to sterilize the segments.

Preoperative fluoroscopic cystography and urodynamic studies were performed in all animals. Under general anesthesia, five beagles underwent cruciate cystotomies on the bladder dome. Augmentation cystoplasty was performed with the allogenic bladder submucosa. A single layer of continuous interlocking sutures with 4-0 vicryl was used for anastomosis. 5-0 Nylon nonabsorbable sutures were placed at the four surgical corners as markers. The augmented bladders were covered with omentum. Cystostomy catheters were used for urinary diversion for 10 to 14 days. Urodynamic studies and fluoroscopic cystography were performed in all dogs at one, two and three months post-operatively. The augmented bladders were retrieved two and three months after augmentation and examined grossly and histologically with hematoxylin and eosin stains.

Results

During the duration of the study, none of the dogs demonstrated any untoward effects. All animals survived until the time of sacrifice without any noticeable complications such as urinary tract infection or calculi formation. Fluoroscopic cystography of all the augmented bladders showed a normal bladder configuration without any leakage at one, two and three months after the procedure.

Bladders augmented with the cell-free allogenic bladder submucosa showed an average increase in capacity of 30%. All animals showed a normal bladder compliance as evidenced by the urodynamic studies. However, some contraction of the graft was observed over time.

At retrieval, the augmented bladders appeared grossly normal without any evidence of diverticular formation in the region of the graft. The thickness of the grafted segment was similar to that of the native bladder tissue. There was no evidence of adhesion or fibrosis. Histologically, the implanted bladder submucosa showed a normal configuration consisting of a urothelial lined lumen surrounded by overlying submucosa and muscle. Migration and infiltration of urothelial and muscle cells from native tissue to a foreign scaffold, such as Teflon, silicone, dura, SIS or bladder submucosa, has been well described. In all retrieved bladders, a urothelial lined lumen was surrounded by submucosal tissue and smooth muscle. An angiogenic response was evident in all specimens.

The results show that isolated bladder submucosa can be used for bladder augmentation, and can become assimilated into the host tissue. However, some contraction may occur over time.

Those skilled in the art will recognize, or be able to ascertain using no more than routine experimentation, numerous equivalents to the specific procedures described herein. Such equivalents are considered to be within the scope of this invention and are covered by the following claims. The contents of all publications and patent applications cited herein are hereby incorporated by reference. Other embodiments are within the following claims.

What is claimed is:

1. A method for surgically augmenting a tissue of a subject, the method comprising augmenting the tissue of the subject with isolated bladder submucosa.

2. The method of claim 1, wherein the isolated bladder submucosa is substantially free of cells.

3. The method of claim 1, wherein the tissue of the subject is bladder tissue.

4. The method of claim 1, wherein the isolated bladder submucosa is allogenic bladder submucosa.

5. The method of claim 1, wherein the subject is a human.

6. The method of claim 1, wherein the isolated bladder submucosa is xenogenic bladder submucosa.

7. The method of claim 1, wherein the step of augmenting the tissue of the subject comprises surgically grafting the isolated bladder submucosa to the tissue of the subject.

8. The method of claim 1, wherein the isolated bladder submucosa further comprises a growth factor for promoting growth of the tissue.

9. A method for increasing bladder capacity of a subject having a bladder, the method comprising surgically grafting isolated bladder submucosa to the bladder of the subject, such that the bladder capacity is increased.

10. A material for tissue reconstruction or augmentation, the material comprising isolated bladder submucosa.

11. The material of claim 10, further comprising an effective amount of a growth factor.

12. The material of claim 10, wherein the material is obtained by microdissection of bladder tissue.
